(19) United States
(12) Patent Application Publication
(10) Pub. No.: US 2025/0276928 A1
(43) Pub. Date: Sep. 4, 2025

(54) A METHOD FOR TREATING AQUEOUS EFFLUENTS WITH ORGANIC CONTENT, PARTICULARLY OIL MILL EFFLUENTS

(71) Applicant: Archimede S.r.l., Caltanissetta (IT)

(72) Inventors: Gianluca TUMMINELLI, Caltanissetta (IT); Gaetano TUZZOLINO, Caltanissetta (IT); Claudio SCARANTINO, Caltanissetta (IT); Calogero GATTUSO, Caltanissetta (IT); Roberto RIZZO, Caltanissetta (IT); Fabio SANTORO, Caltanissetta (IT)

(21) Appl. No.: 18/862,339

(22) PCT Filed: May 4, 2023
(86) PCT No.: PCT/IB2023/054635

§ 371 Date: Nov. 1, 2024

(30) Foreign Application Priority Data
May 4, 2022 (IT) 102022000009149

(51) Int. Cl. C02F 11/08; C02F 101/30; C02F 103/32

(52) U.S. Cl. CPC C02F 11/086; C02F 2101/30; C02F 2103/322; C02F 2301/066; C02F 2305/00

(57) ABSTRACT

Disclosed herein is a method for treating aqueous effluents with organic content, particularly oil mill effluents, that includes: providing a flow of aqueous effluents with organic content, pressurizing the flow of aqueous effluents to a pressure value equal to or greater than the pressure value at the critical point of water, and heating the flow of aqueous effluents to a temperature value equal to or greater than the temperature value at the critical point of the water, processing the pressurized and heated flow of aqueous effluents by means of a supercritical water reactor, and subjecting the aqueous effluents processed by means of the supercritical water reactor to a catalytic hydrogenation treatment.

### FIELD OF THE INVENTION

The present invention relates to the treatment of urban or industrial effluents, specifically from the food processing industry. The invention was developed with particular, but not exclusive, reference to the aqueous effluents from oil mills.

### STATE OF THE ART

The waste aqueous effluents of oil mills mainly comprise the vegetation water of olives, which is intrinsically contained in the fruits of olive trees up to 50% by weight, and the water which is added during the extraction process.

The amount of waste water which is produced depends on the extraction process which is implemented: for example, a three-stage centrifugation generates an amount of water which is at least twice as much as the amount of oil produced, while the two-stage treatment generates the lowest amount of waste (approximately equalling 10% of the mass of the processed olives).

Also the waste composition strongly depends on the process technology employed, but on average it consists of 80% water, 15-18% organic compounds (mainly sugars) and approximately 2% inorganic salts. The solution pH is generally lower than 5.5, and the chemical oxygen demand (COD) may reach as high as 200000 ppm; this is an extremely high value which, together with the presence of rather stable phenolic compounds, makes the waste water of oils mills a particularly problematic production residue from the point of view of both health and environment, due to the high toxicity thereof for plants and animals.

The membrane-based processes in the treatment of aqueous effluents of oil mills offer some advantages as compared with conventional biological, chemical, physical-chemical and thermal processes, i.a., the low specific consumption, the absence of additives and the absence of phase change.

On the other hand, such processes involve technological problems due to the phenomenon of concentration polarization and to the soiling of the membranes, caused by the particular composition of the aqueous effluents. Typically, the membrane-based processes involve the combination of various different technologies, characterised by a gradually decreasing characteristic dimension of the filtering element, from the first stage of microfiltration and ultrafiltration (enabling a drastic reduction of suspended solids) to a second stage of nanofiltration and reverse osmosis (enabling a drastic reduction of polluting organic compounds and the recovery of pure water).

A further problem of the aqueous effluents of oil mills consists in the high energy consumption as opposed to a low recycling possibility of the purified effluent. This is essentially made of water which—albeit it may be introduced into the mains—does not enable any substantial conversion or recovery of the energy consumed in the purification process.

### OBJECT OF THE INVENTION

The present invention aims at solving technical problems outlined in the foregoing. Specifically, the invention aims at providing a method for treating aqueous effluents with organic content, e.g., aqueous effluents of oil mills, which is substantially devoid of the technical problems affecting the known treatment technologies outlined in the foregoing, particularly the membrane-based treatment technologies, and which moreover enables the recovery of aqueous effluent by means of a conversion into products adapted to be further utilized.

### SUMMARY OF THE INVENTION

The object of the invention is achieved by a method having the features forming the subject of the claims that follow, which form an integral part of the technical disclosure provided herein in relation to the invention.

### BRIEF DESCRIPTION OF THE FIGURES

The invention will now be described with reference to the annexed Figures, which are provided by way of non-limiting example only and wherein:

FIG. 1 is a schematic representation, by means of a block diagram, of a first embodiment of the treatment method according to the invention,

FIG. 2 is a schematic representation, by means of a block diagram, of a second embodiment of the treatment method according to the invention, and

FIG. 3 is a schematic representation, by means of a block diagram, of further method steps according to the invention.

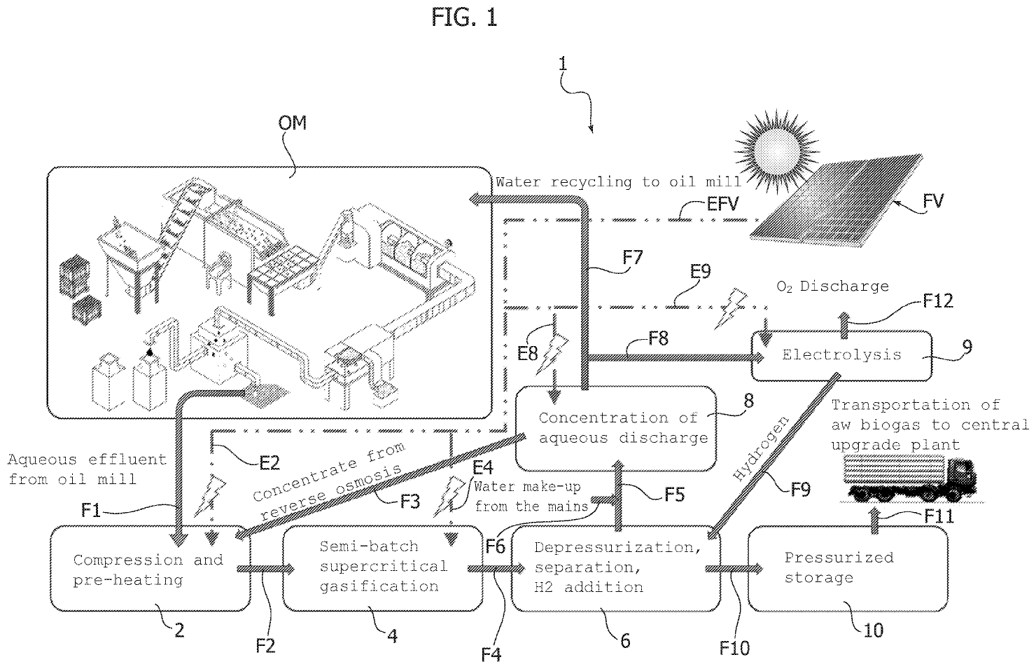

The diagrams of FIGS. 1 to 3 are accompanied, in addition to the usual reference numbers which consistently appear throughout the description, by short text which portions only aim at helping understand each diagram with reference to some specific embodiments (which, however, are extensively detailed in the description). Such text portions—as will be evident from the following description—must not be interpreted in any way as constituting information or limiting elements with reference to the description of the invention or the object of the diagram, wherein the reference is always represented by the detailed description provided in the following.

### DETAILED DESCRIPTION

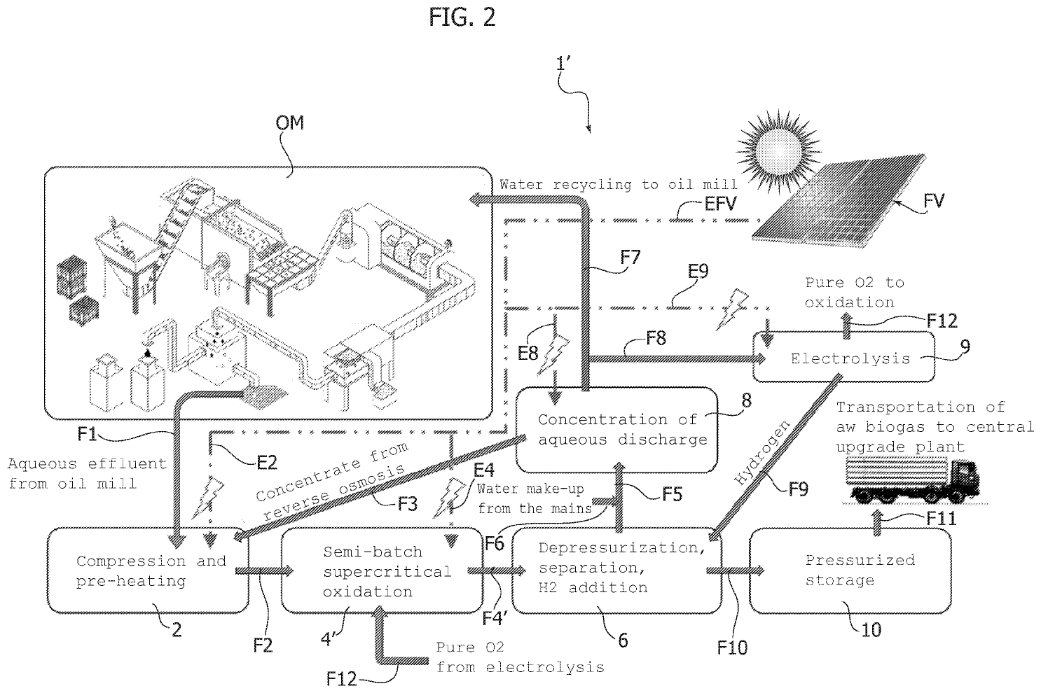

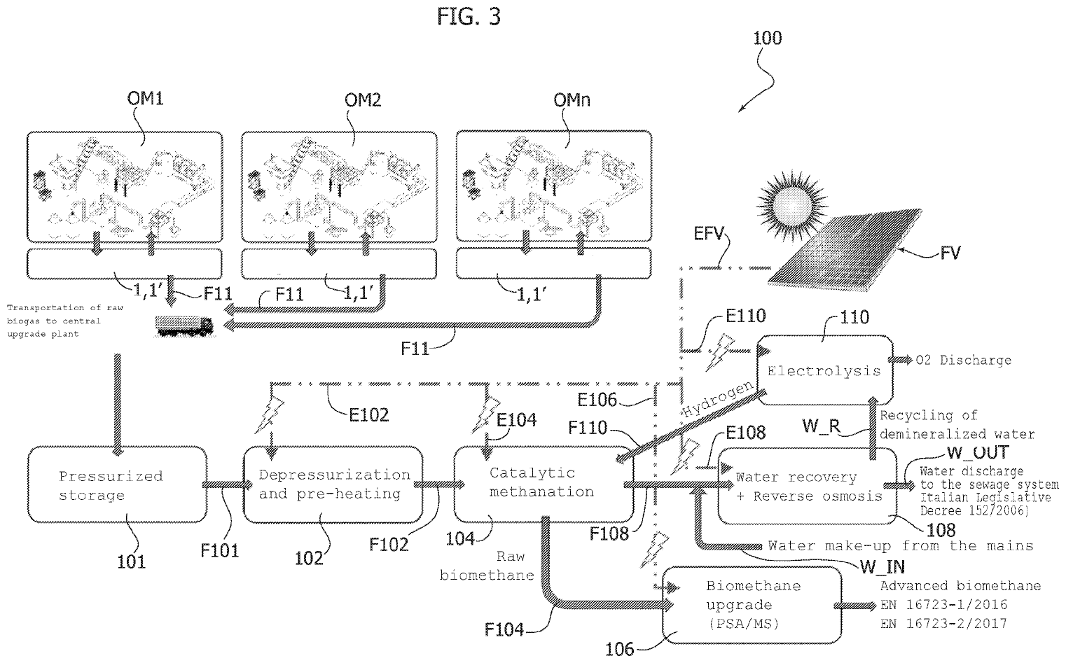

As a general foreword, the treatment method according to the invention comprises two treatment sub-methods, one (as per FIGS. 1 and 2) for the treatment in supercritical conditions of a flow of aqueous effluents with organic content (e.g. effluents from an oil mill, but it is intended that other activities are comprised, such as e.g. dairies and the related aqueous effluents, as well as urban effluents, leachates etc.) for converting the complex organic molecules into simpler molecules and hydrogen, the other (as per FIG. 3) for a further conversion of the products of the supercritical treatment into combustible products by means of catalytic hydrogenation of carbon oxides (e.g. catalytic methanation), preferably with a subsequent upgrade in order to let the product meet the requirements demanded by regulations for the use as automotive fuel or the input into distribution mains.

The method according to the invention envisages, i.a., an integrated management of both sub-methods for treating aqueous effluents with organic content: both sub-methods are not only complementary on a functional plane, but they may be applied both on a single territorial level (preferably without interruptions from one sub-method to another) and on two territorial levels.

Specifically, referring to the pair of territorial levels, considering a “micro” geographic scale (territories of the single municipalities and small groups of municipalities), the first sub-method (FIG. 1 or 2) is well adapted to be used in the proximity of small to medium sized single local olive processing plants (with reference, by way of non-limiting example only, to an oil mill), for on-site treatment of the aqueous effluents produced locally and for the local production of raw syngas, by means of a thermo-chemical supercritical gasification process (again, preferably with semi-batch operation, to better adapt to the flow typology of effluents coming from an oil mill or a dairy, but also being adapted to work in continuous operation).

The first sub-method is therefore performed in a delocalized and decentralized fashion, as against the second sub-method, which is applied jointly and in parallel with the first on a “macro” geographical scale (on a county or intercounty territorial level, up to a regional scale).

The second sub-method involves the collection and a catalytic hydrogenation treatment (which may or may not be centralized), specifically of carbon oxides (briefly, in the following, “catalytic hydrogenation”) e.g., by means of the technology of catalytic methanation, of all the raw syngas produced in the delocalized sites. The catalytic hydrogenation treats the carbon oxides (mainly monoxides and dioxides), totally or nearly totally, for the production of (bio) fuels.

The catalytic hydrogenation treatment comprises a wide range of specific treatments according to the final product required. If the required final product is methane, the catalytic hydrogenation treatment comprises catalytic methanation. If the required final product is gasoline or gasoil, the catalytic hydrogenation treatment is a Fischer-Tropsch process.

According to an advantageous aspect of the invention, through the method, both for delocalized plants and for a centralized plant (and generally speaking for both sub-methods), a significant part of, or even all the electrical energy needed, which is destined nearly exclusively to the compression of the supply flows and to the support of the operating conditions in a supercritical water reactor, comes from a renewable energy source, preferably a photovoltaic field. It is therefore possible to obtain, as a final product, a biofuel which is a so-called “e-fuel”, i.e., a fuel obtained through an energy input from a renewable source.

Specifically, a photovoltaic field with a limited peak power, which may be already existing or newly installed at each of the selected oil mills (or local sites), coupled with energy an backup system (electrical storage batteries), may provide for the energy needs of the local plant, while keeping the possibility of tapping into the national mains in case more energy is needed. In any case, embodiments are possible wherein the photovoltaic field is of the so-called “stand alone” type, i.e., totally independent from the electric mains and only operating in conjunction with an electrical energy storage system, such as a battery pack. In this way an important economic advantage is achieved, because it is no longer necessary to pay the distribution excise duties and charges.

The same thing may be said with reference to the second sub-method and to the related centralized plant. In this regard, the second sub-method may be implemented within the same plant which performs the first sub-method, therefore envisaging, in this case as well, a local or “micro” scale, or else it can b implemented in a plant which is physically distant and dislocated to another geographic area, and/or operating on a different (“macro”) scale. In the latter case, the syngas produced with the first sub-method is input into pressurized cylinders, and is transferred to the site where the second sub-method is implemented, by means of cylinder wagons which collect the cylinders from the single oil mills, dairies or generally production sites which release aqueous effluents with organic content.

A preferred goal of the second sub-method is, i.a., the centralized upgrade with the production of fuels which comply with the law regulations for automotive use, or for the input into the national mains as natural gas (in the case of advanced biomethane). As already remarked in the foregoing, the produced fuels comprise biofuels and e-fuels, according to the energy input which is used.

As will become clear in the following, in order to enrich the syngas obtained from the supercritical water treatments, especially from the supercritical water gasification treatment, so as to produce a fuel (e.g., an advanced renewable fuel), the second sub-method envisages a catalytic hydrogenation treatment, which requires the supply of hydrogen as a second process reagent.

According to an advantageous aspect of the invention, the hydrogen required is preferably of the so-called “green” type (green hydrogen), i.e., a hydrogen which is obtained from a renewable source. In this case, the renewable electrical energy produced by the photovoltaic solar field, coupled with the plant implementing the second sub-method, may be destined almost exclusively to the supply of a system of electrolysers and storage compressors. This enables the centralized production of green hydrogen from the electrolysis of water by means of solar energy, and the temporary storage thereof for subsequent uses (in case of insufficient solar irradiation).

In preferred embodiments of the invention, and referring to aqueous effluents having organic content and originating from oil mills, the combination of both sub-methods is adapted to treat semi-continuously, throughout an “operating year” of 2000-3000 rated operation hours, the amount of aqueous effluent produced by one or more oil mills in the two operation months which are typical thereof. Of course, the method according to the invention may be used for a continuous treatment or for treatments distributed in time, so that, in the latter case, the equipment may be downsized.

In other words, a plant operating semi-continuously (or in semi-batch mode) must be sized so as to manage the treatment needs of the flow of effluents coming from a source which in turn operates semi-continuously. This means that the sizing of the equipment and of the filtration systems is necessarily bigger, as it is impossible to provide a deferred and distributed treatment of the effluents with lower flow rates and the storage of the flow rate in excess.

In the case of plants operating continuously, the sizing of the equipment and of the filtration systems may be smaller than in the case of a semi-continuous operation mode; in other words, the system may be sized for operating with smaller effluent flows: in this case, the flow rate in excess may be stored and treated in a deferred and distributed fashion in time (e.g., if an oil mill operates for only two months a year, the plant may operate for twelve months a year, by distributing over twelve months the treatment of the effluent generated in two months by the oil mill), and when the oil mill (or the source of aqueous effluents) is not operating, hydrogen may be stored in tanks and/or electrical energy may be stored in batteries (preferably with priority to the former solution), so as to ensure the availability of short autonomy periods when solar irradiation is absent and/or in the case of temporary flow rate peaks of the aqueous effluents which are destined to undergo the treatment.

The storage of electrical energy is preferably of secondary importance with respect to the storage of hydrogen, which is generally considered as a priority: when the storage tanks are full, then the accumulation of electrical energy is started.

In any case, management solutions wherein the reactor (and, generally speaking, the plant) operates on a continuous basis enable minimizing the size of the equipment, distributing duty cycles and avoiding repeated on-off switching. The limits of the continuous operation will only depend on the availability of small-size equipment on an industrial scale.

Referring to FIGS. 1 and 2, there will now be described two embodiments of the first sub-method, with reference to the simplified block diagrams provided in said Figures.

Referring to FIG. 1, reference 1 generally denotes the diagram of a first embodiment of the first sub-method, which is part of the general method according to the invention. The main feature of sub-method 1 is the supercritical water oxidation treatment.

In the following description, the sub-method 1 is applied to an oil mill OM, by intercepting a flow F1 of aqueous effluents with organic content produced therein due to the normal processing of olives. Of course, the sub-method 1 may be applied to any activity, of industrial or other nature, which generates a flow of aqueous effluents with organic content.

As a general rule, each of the blocks which will be described in the following must be understood as representative both of a method step and of a device operating within a plant which implements the sub-method.

The flow F1 is subjected to a treatment which raises the pressure and the temperature thereof, and which is represented by a block 2. By means of a compression and pre-heating system, the flow F1 acquires conditions of pressure and temperature which are respectively higher than the pressure and the temperature at the critical point of water (221 bar and 374° C.), preferably acquiring a rated pressure of 240 bar and a rated temperature of about 600° C. (both being clearly higher than the values of the critical point of water).

The flow F1 thus processed is associated, for easiness of description, to a new reference F2. It must be borne in mind that the flow F2 may completely correspond to the flow F1, apart from the different thermo-dynamic conditions, but it generally corresponds to the sum of the flow F1 with a recirculation flow F3, the composition whereof will be described in the following.

Flow F2 is subsequently sent to a supercritical water treatment 4, which in this embodiment corresponds to a supercritical water gasification treatment, means of a implemented by supercritical water gasification reactor. It is important to remark that the water required for the treatment is the same water contained in the aqueous effluent constituting the flow F2 and, as a consequence, the flow F2.

The gasification reactor preferably operates in a semi-batch mode, because typically also the processing of olives operates by batches.

In the gasification reactor the reagent mixture, i.e., the flow F2, is kept at the same temperature and pressure for a residence time which maximises the conversion of the organic components and the yield of methane, which is the most important component for the following treatments. These treatments also involve hydrogen, which is formed in the mixture during the gasification process.

In the processes of thermal (non-catalytic) gasification in an aqueous supercritical environment, which are widely described in the related literature, the residence time in the reactor is typically between 100 seconds and a few tens of minutes.

The conditions of temperature, pressure and residence time are chosen—in a way known in itself—in such a way that, when the chemical composition of the processed flow F2 changes, it is possible to obtain a gaseous product characterized by a maximum hydrogen yield or by a given ratio between hydrogen and carbon oxides, irrespective of the methane yield.

As a matter of fact, the latter condition is potentially more advantageous, because it enables saving “green” hydrogen in order to comply with the purity requirements of methane, which are the goal of the second sub-method.

Reference F4 exiting block 4 identifies the flow of products of the supercritical water (gasification, in this embodiment) treatment, which substantially corresponds, apart from negligible internal leaks, to the flow F2.

The flow F4, once it has left the reactor, is depressurized (block 6) to an intermediate level of pressure, which generally amounts to 40-60 bar, it is cooled and into separated a gaseous fraction corresponding to the intermediate product of interest (i.e., wet syngas) and a into liquid fraction, comprising an aqueous flow with organic residues which are neither completely oxidized and dissolved in water, or converted into gas because of kinetic or thermodynamic hurdles.

The liquid fraction (flow F5), extracted at a pressure level generally amounting to 40-60 bar, is subjected to filtration (concentration, block 8) by means of a conventional membrane separation system. The choice of the type and of the number of separation stages (microfiltration, nanofiltration, ultrafiltration, reverse osmosis) is made, with known criteria, in such a way as to implement a configuration the problems of polarization, which minimizes obstruction and soiling of the membranes, and in such a way as to support the operations of regeneration and restoration of the filtering system.

A preferable condition for a satisfactory operation of the filtering system, wherein the last stage is performed by means of reverse osmosis, is characterized by the absence of suspended solids in an appreciable quantity, or anyway in a quantity which is incompatible with the membrane system.

In this way it is possible to take advantage of the fact that the flow F5 is already separated in pressurized conditions and is ready to be supplied to the reverse osmosis modules, with a consequent energy saving for the pumping process. In this context, it may be necessary to add make-up water from the mains (flow F6) before the input into the reverse osmosis unit, in such a way as to produce at the same time an amount of demineralized water which may be useful for other purposes, e.g., for the recirculation to the oil mill OM, flow F7, or for the supply to an electrolyser 9 (flow F8) for the production of “green” hydrogen, as will be described in the following.

Moreover, the (aqueous) flow of concentrated solid residue (flow F3, mentioned in the foregoing) produced by means of reverse osmosis, may reach, after each concentration cycle, concentrations of organic residues which are 3-4 times as high as in flow F1, and may therefore be recirculated into the supercritical gasification reactor (block 4) as it contains unreacted compounds.

The single recirculation operation may be repeated more than once for each “fresh” effluent batch F1 from the oil mill, so as to maximize the conversion of the organic content into light gaseous species. The whole process may be schematized, substantially, as a semi-batch process, because of the presence of a first initial batch of effluent (flow F1) which is processed, separated and partially recycled (only as regards the concentrated residue of reverse osmosis, batch F5): such operations take place without interruptions, with the final effect of lengthening the total residence time in the reactor (or, rather, of the total time during which the reactor is kept at the same temperature), although at each recirculation the real average composition of the flow (F1+F3) entering the reactor may be different—the concentration of organic residue becoming increasingly high or being kept constant by diluting the concentrate F3 to the operational concentration of the gasification reactor (typically, effluent:water=1:10, in any case not higher than 1:5). A booster pump is preferably provided for the recirculation of flow F3, because an unsupported circulation would require pressure conditions which are incompatible with filtration.

The gaseous fraction of flow F4, which is denoted by reference F10, is added with pure hydrogen (flow F9, in an amount sufficient to reach a volume proportion of 4:1 with respect to the carbon dioxide which is already present in fraction F10), which in the meantime has been produced by means of a hydrolysis process of demineralized water by the electrolyser 9. The resulting mixture is temporarily stored under pressure in cylinders (block 10), before being transferred to cylinder wagons (F11) and being transported to the central enriching (catalytic hydrogenation)/upgrade site, where the second sub-method is implemented.

The oxygen which has been simultaneously produced by the electrolyser 9 may be let out into the atmosphere without needing purification (flow F12).

According to an advantageous aspect of the invention, the energy requirement of the plant implementing method 1 may be met by means of a photovoltaic plant (or field) FV, preferably equipped with an electric storage battery.

References E2, E4, E8, E9 identify the flow of the electrical energy respectively supplied to the devices of stages 2 (compression and pre-heating), 4 (supercritical water gasification treatment), 8 (concentration/filtration of the aqueous fraction of flow F4), 9 (electrolyser). Each flow E2, E4, E8, E9 is a part of a general flow of electrical energy EFV coming from the photovoltaic field FV, which may also supply a flow of electrical energy for recharging the electric accumulators. Actually, when the gasification reactor is not operating, the electrical energy produced by the photovoltaic field FV may be destined to recharging the storage batteries, i.e., supplying the user devices of the oil mill OM.

Generally speaking, it is not necessary to supply all the method stages mentioned in the foregoing with the flow of electrical energy EFV coming from the photovoltaic field FV, but preferably at least one (or at least the one demanding more energy, such as electrolyser 9) should be supplied with it, and even more preferably all the stages should (i.e., all flows E2, E4, E8, E9 should be supplied).

Referring to FIG. 2, reference 1′ generally denotes a second embodiment of the first sub-method according to the invention. The corresponding block diagram, and generally speaking the sub-method, is identical to the sub-method described with reference to FIG. 1, except for the different supercritical water treatment. Specifically, instead of a supercritical water gasification treatment, and of the corresponding reactor, there is envisaged a supercritical water oxidation treatment. For this reason, the block 4 in FIG. 1 is replaced with a block 4′ in FIG. 2 (the electrical supply from the photovoltaic field FV is consequently renamed as E4′, in the same way as the flow exiting the supercritical water treatment is renamed as F4′. The further differences will be described in the following, but generally speaking the description referring to FIG. 1 still applies (and will not be repeated in its entirety), and all the references previously adopted for FIG. 1 are to be deemed as having the same meaning (if not explicitly stated otherwise).

In the sub-method of FIG. 2, the syngas produced in the delocalized sites (flow F10) is expected to be composed almost exclusively of pure carbon dioxide and hydrogen; it requires a higher amount of electrolysis-derived “green” hydrogen, but also leads to a higher pureness of the final product.

In this case, the supercritical water oxidation treatment of the organic species which are present in the vegetable water and in the aqueous effluents of the oil mill (flow F1) takes place with a direct reaction at a rated pressure of 240 bar and a rated temperature of approximately 580°, with pure oxygen or compressed air. The flow F1 is preferably brought to corresponding or slightly lower pressure values during the heating and compression step of block 2. Again, it will be remarked that such temperature and pressure levels are perfectly within the water supercritical field.

If pure oxygen is used, as may be seen in FIG. 2, oxygen may be provided directly from electrolyser 9 (flow F12, which may optionally be supplied to block 2 together with flow F1), which is already present and is employed for the production of hydrogen; in this case, however, the electrolyser 9 will have to be provided with a powerful, high-performance purification (dehydrogenation) section for the oxygen which, in order to be compressed safely and to be supplied to reactor 4′, must be totally devoid of hydrogen traces.

The main advantage of such a solution consists in obtaining a syngas F10 having a composition which is almost independent from the nature of the organic components of the supplied vegetable water, because it is expected to comprise substantially pure carbon dioxide and hydrogen.

On the other hand, because the amount of CO2 which is present in the main mixture F4′ is higher, in absolute terms, than the amount produced in the case of described the previously supercritical water gasification treatment, it will be necessary to add a higher amount of “green” hydrogen derived from electrolysis, in order to comply with the volume ratio of 4:1 input into the enriching (catalytic hydrogenation)/upgrade plant.

Nevertheless, the quality of the advanced renewable biomethane obtained after the methanation treatment will be higher and certainly more compliant with the technical specifications imposed by the current regulations, because the product will be devoid of hydrocarbon components having two or more carbon atoms (ethane, propane, butane and higher).

Referring to FIG. 3, there will now be described the second sub-method according to the invention, identified by reference 100. Such sub-method achieves enriching (catalytic hydrogenation) and, if desired, a centralized upgrade of the syngas (F10) coming from the local plants installed in the vicinity of the olive processing sites, which are herein identified by the references OM1, OM2, OMn (the latter identifying an nth oil mill).

The second sub-method substantially comprises a catalytic hydrogenation (e.g., a catalytic methanation) of the carbon oxides present in the syngas, which was already previously added with “green” hydrogen, again obtained by electrolysis of demineralized water by means of photovoltaic solar energy. Moreover, it must be borne in mind that the central enriching/upgrading site may be equipped with an electrolyser of its own, in order to produce further hydrogen to be used for make-up operations.

Referring to FIG. 3, in a preferred embodiment the sub-method for enriching and upgrading the syngas to obtain biofuel (or, generally speaking, to an “e-fuel” of biological origin, according to the catalyst used for hydrogenation), for example methane which may be used for distribution in the national gas mains (regulation UNI EN 16723-1/2016) or for automotive purposes (regulation UNI EN 16723-2/2017), is performed in a centralized site, which receives the batches of syngas produced in the “local” sites distributed over the territory, wherein the first sub-methods are implemented. The type of treatment which is implemented on a local level—oxidation or gasification—depends on the residual organic content, on whether it is more or less resistant to gasification, and on the species (advanced fuel or “e-fuel”) the obtention whereof is desired.

For example, if the purpose is to obtain advanced biomethane or LPG, the preference will go to gasification, provided that the organic content of the aqueous effluent is easily gasifiable. On the other hand, if the purpose is obtaining DME (dimethyl ether), gasolines, gasoil etc., the preference will go to oxidation, combined with a catalytic hydrogenation reactor with a correspondingly selective catalyst. This applies also if the purpose is to obtain pure methane, without the need to separate the GPL fraction, as is the case for syngas from supercritical water gasification.

In the latter case the preference will go to oxidation, although it requires a higher input of resources (especially hydrogen), yielding nevertheless a purer product.

The double management on which the method according to the invention is based, which takes place both in a centralized fashion (second sub-method) and in a delocalized fashion (first sub-method, whatever the embodiment may be), may enable the central enriching and upgrading plant to accept as input (flow F10, possibly stored in cylinders) charges of syngas or anyway of gas mixtures having a high content of carbon oxides, or even flows of stored carbon dioxide coming from other sources or from plants other than oil mills.

In any case, the centralized plant is designed to operate continuously, unlike the delocalized plants which, as mentioned in the foregoing, are preferably designed to operate semi-continuously, with recirculation (semi-batch operation).

The syngas batches transported by the cylinder wagons, once they have been accepted after a sampling procedure to check the correct volume ration between hydrogen and carbon dioxide, are temporarily stored under pressure in tanks (block 101).

From these tanks the substances are withdrawn, with a known and constant flowrate F101, in order to be pre-heated (block 102, preferably after depressurization) and to be subjected to a catalytic hydrogenation treatment 104, which in the embodiment shown in the Figure is a catalytic methanation treatment performed by means of a methanation catalytic reactor.

During a catalytic methanation treatment, due to the permanence at high temperature (300-350° C.) in the presence of a catalyst, typically based on nickel or ruthenium supported on alumina or mixed aluminium and magnesium oxides (each of which having peculiar properties of activity and selectivity in methane), the carbon monoxide and dioxide, which are abundantly present in the syngas, participate in the water gas shift reaction and in the methanation reaction.

The former is a well-known reaction of chemical balance between carbon monoxide and water, on one hand, and carbon dioxide and hydrogen, on the other hand:

CO+H₂O⇄CO₂+H₂

Such a reaction is exothermic (therefore the balance towards the desired product is favoured by a low temperature), and it generally takes place in one or more reactors other than the methanation reactor, with different catalytic systems and operating conditions, in order to maximize the amount of hydrogen in the mixture (flow F102) before the entry thereof into the methanation reactor and before the contact with the nickel/ruthenium catalyst.

The second reaction leads to the production of methane and water starting from carbon dioxide and hydrogen, practically involving a reaction of carbon reduction from the oxidation state +4 to the state −4:

CO₂+4H₂→CH₄+2H₂O

The amount of produced methane, for given supply and reaction conditions, depends on the activity and the selectivity of the catalyst, as well as from the yield thereof in time and space. A good catalyst must preferably exhibit high activity, even at comparatively low temperatures, which is also useful in order to maximize the yield at reaction equilibrium, because the process is exothermic, a high and must exhibit selectivity towards the desired compound. In the case of the methanation process, the function of the catalyst is to orient the reaction towards the preferential production of methane instead of other higher hydrocarbons (ethane, propane) by means of the so-called kinetic control of the reaction.

In order to tackle with the high exothermy of the reaction, a possible operational choice is to operate in high excess of either one of the reagents (hydrogen or carbon oxides): in this case the need will arise of a plurality of methanation stages, interspersed with the cooling of the reacting mixture, with the removal of the water possibly produced in the reaction and with the make-up of the reagent consumed, in order to restore the stoichiometric excess.

At the exit of the catalytic reactor, the gaseous mixture is cooled paying particular attention to energy recovery (thermal integration among the flows, in order to favour pre-heating of the supply flow at block 102), which in any case plays a vital role in the global economy of the process.

The combination of the methanation reaction and of the reaction of removal of gas from water shows, purely theoretically and neglecting the other possible concurrent reactions (such as i.e., the formation of coke deposits, which has a detrimental effect on the catalytic system), that the global process of conversion of the carbon oxides into methane takes place with a net consumption of hydrogen and a net formation of water.

After cooling, the biomethane gaseous mixture is separated from the aqueous fraction contained therein so as to isolate a gaseous flow F104 which is to be subjected to upgrade (block 106, generally optional), if the produced biomethane is to be used for civil uses or for automotive uses. In this regard, flow F104 corresponds to a raw biofuel, which may constitute a final product for the method according to the invention; of course, if a specific biofuel, i.e., an advanced biofuel, is desired, the upgrade of block 106 is necessary. The aqueous solution forms a flow F108, which is subjected to a treatment for water recovery and reverse osmosis filtration, block 108.

In detail, the condensed water is removed by means of simple phase separation, and it is subjected to a reverse osmosis treatment in order to obtain demineralized water. Such water is recirculated to an electrolyser 110 combined with the plant implementing the second sub-method (enriching/upgrade plant); the electrolyser 110 is used for the production of “green” hydrogen, which in this case acts as a make-up for maintaining the stoichiometric excess in the methanation plant (flow F110).

The water needed for the electrolysis process must be demineralized: for this reason, the plant which implements method 100 may be equipped with a pre-treatment (reverse osmosis) unit for the water from the mains, if it is freshly supplied to the electrolyser, and for the treatment of the water generated in the methanation reactions.

In this regard, although the global process leads to a net production of water, and therefore may be self-sustaining, a more reasonable choice which is convenient in the global economy does not imply the use of a certain amount of fresh water from the mains (flow W_IN) for cooling the syngas mixture exiting the methanation reactor, so as to have, as an input to the following reverse osmosis treatment 108, an already diluted solution (as shown in FIG. 3). This would produce a flow of demineralized water W_R to supply to the electrolyser, and a concentrate which could anyway comply with the specifications concerning a direct discharge into the sewage system (flow W_OUT), according to the Italian Legislative Decree 152/2006.

The biofuel or e-fuel (biomethane, in the embodiment described in FIG. 3) separated from the mixture exiting the catalytic hydrogenation reactor (in this case, methanation, flow F104) contains a small percentage of water vapour, and therefore must be subjected to dehumidification if the purpose is to comply with the limits imposed by the regulations mentioned in the foregoing (UNI EN 16723-1/2016 and UNI EN 16723-2/2017).

Moreover, always with the purpose of complying with such regulations, it is necessary to remove unreacted hydrogen from the mixture, and to this end two possibilities may be taken into consideration:
in the case of operating with an excess of hydrogen, the carbon oxides will be totally converted, and it will be necessary to separate the unreacted hydrogen from biomethane (for example by means of the Pressure Swing Adsorption, PSA, technology), so as to comply with the maximum limits of 0.5% and of 2% in volume, respectively, required by regulation UNI EN 16723-1 for the input into the mains, and UNI EN 16723-2 for automotive uses;
in the case of operating with an excess of carbon oxides, hydrogen and—very probably—also carbon monoxide are completely converted, and it is necessary to separate the unreacted carbon dioxide from biomethane (e.g. by means of the membrane separation technology), so as to comply with the maximum limit of 2.5% in volume imposed by both regulations mentioned in the foregoing.

A system for the final compression of biomethane to be input into the automotive consume network or into the national distribution mains, after an adjustment of additives and odorants added to the biomethane, completes the method 100. The latter operation is however optional, because in embodiments it is possible to obtain, as a product of the method according to the invention, only the biofuel of flow F104, therefore implementing the catalytic hydrogenation only, without necessarily performing further treatments for the compliance with specific regulations.

Moreover, as has been stated in the foregoing, it is not strictly necessary for methods 1 and 1′ to take place in separate sites: in some embodiments, both methods may be implemented in a single place, by supplying flow F4 directly to the catalytic methanation treatment of block 104 or by supplying flow F10 directly to the catalytic hydrogenation treatment of block 104. In this case, the addition of hydrogen to flow F10, i.e., the flow F9 coming from electrolyser 9, may conveniently be adjusted so as to obtain the desired stoichiometric ratio (or a slightly higher ratio, with an excess of hydrogen) in order to produce the advanced biofuel desired as a final product. The possible presence of oxygen in excess may be vented to the atmosphere without the need of a further purification. This is true for both supercritical water treatments of sub-methods 1, 1′ (oxidation and gasification), but in the case of supercritical water oxidation the discharge of oxygen into the atmosphere takes place only when the biofuel required as a final product requires an amount of hydrogen involving a production of oxygen, by electrolyser 9, higher than needed for oxidation only.

As already remarked in the foregoing, the biofuel produced by means of catalytic hydrogenation may be or may not be subjected, according to needs, to an upgrade for achieving compliance with regulations.

As already anticipated, according to an advantageous aspect of the invention, the hydrogen needed for the methanation process preferably comes from a renewable energy source, e.g., a photovoltaic solar plant. To this end, electrical energy may be drawn from a photovoltaic field FV installed in proximity of the plant which implements sub-method 100.

The use of hydrogen produced from a renewable source enables the whole plant to produce so called e-fuels, which benefit from incentives of the Energy Services Manager according to the current regulations.

References E102, E104, E106, E108, E110 denote the flow of the electrical energy respectively supplied to the devices of stages 102 (depressurization and pre-heating of the syngas), 104 (catalytic methanation treatment), 106 (biomethane upgrade), 108 (recovery of water from the biomethane gaseous mixture), 110 (electrolyser). Each flow E102, E104, E106, E108, E110) is a part of the general flow coming from the photovoltaic field FV, wherefrom also a flow of electrical energy may be derived for recharging electric accumulators. In the periods when the gasification reactor is not in operation, the electrical energy produced by the photovoltaic field FV may actually be destined to recharging the storage batteries.

Generally speaking, it is not necessary for all method steps mentioned in the foregoing to be supplied with the flow of electrical energy EFV coming from the photovoltaic field FV, but preferably at least one of them (or at least the one which consumes most energy, e.g. electrolyser 110) is supplied with it, and even more preferably all of them are (i.e., the photovoltaic field supplies all the flows E102, E104, E108, E106, E110).

Moreover, the flow of electrical energy EFV coming from the photovoltaic field FV may be used to supply a battery of electrolysers 110, which generate hydrogen by means of electrolytic splitting of the water molecules. This solution may be an alternative to storing energy from the photovoltaic field in batteries, by preferring a direct use by hydrogen overproduction and storage thereof in pressurized cylinders.

It will be appreciated, therefore, that the global method 1, 1′ and 100 according to the invention enables without any technical treating, effectively and disadvantages, the aqueous effluents with organic content from any source, by recycling the effluents by means of conversion into species for further use, such as “raw” bio-fuel/e-fuel or as a specific biofuel for automotive uses or for the input into the mains (if it is subjected to upgrade in block 106).

Moreover, the possibility is offered of massively resorting to renewable energy sources, in addition to the option of adapting and scaling the plants which implement methods 1, 1′ and 100 according to the activity which originates the aqueous effluent and according to the territory where the activity is located. In comparison with the treatment methods of the known art, the purification of the flow of aqueous effluents does not depend completely on a filtration by means of membrane separators/concentrators, but it mainly depends on more efficient supercritical water reactors. Therefore, the energy consumption of such devices—when they are sized to process the whole flow—is significantly decreased, and the same is true for the costs of production, installation and maintenance of such devices. The advantage is further increased if the method according to the invention is implemented in a continuous operation mode, since this solution further decreases the size of all plant components, including the membrane separation/concentration devices.

Of course, the implementation details and the embodiments may amply vary with respect to what has been described and illustrated herein, without departing from the scope of the present invention, as defined in the annexed claims.

1. A method for treating aqueous effluents with organic content, comprising:
providing a flow of aqueous effluents with organic content,
pressurizing said flow of aqueous effluents with organic content to a pressure value equal to or greater than a pressure value at a critical point of water, and heating said flow of aqueous effluents with organic content to a temperature value equal to or greater than a temperature value at the critical point of the water,
processing the pressurized and heated flow of aqueous effluents by means of a supercritical water reactor, and
subjecting the aqueous effluents processed by means of the supercritical water reactor to catalytic hydrogenation, including catalytic hydrogenation of carbon oxides.

2. The method according to claim 1, wherein said catalytic hydrogenation comprises a catalytic methanation.

3. The method according to claim 1, further comprising depressurizing the effluents processed by means of the supercritical water reactor prior to said subjecting the aqueous effluents processed by means of the supercritical water reactor to catalytic hydrogenation.

4. The method according to claim 1, further comprising adding hydrogen to the aqueous effluents processed by means of the supercritical water reactor prior to said subjecting the aqueous effluents processed by means of the supercritical water reactor to catalytic hydrogenation.

5. The method according to claim 4, further comprising providing an electrolyzer for generating the hydrogen to be added to the flow of aqueous effluents processed by means of the supercritical water reactor.

6. The method according to claim 5, further comprising separating a gaseous fraction of said aqueous effluents processed by means of the supercritical water reactor from a liquid fraction of said aqueous effluents processed by means of the supercritical water reactor, the gaseous fraction being subjected to said catalytic hydrogenation, the liquid fraction being treated to separate an aqueous fraction from a solid fraction.

7. The method according to claim 6, wherein said electrolyzer is supplied with the aqueous fraction extracted from the liquid fraction of said aqueous effluents processed by means of the supercritical water reactor, and wherein the solid fraction of said aqueous effluents processed by means of the supercritical water reactor is aggregated to said flow of aqueous effluents with organic content supplied to the supercritical water reactor.

8. The method according to claim 1, further comprising providing an electrolyzer for generating hydrogen for the catalytic hydrogenation treatment to which the aqueous effluents processed by means of the supercritical water reactor is subjected.

9. The method according to claim 6, wherein at least one of the supercritical water reactor, the electrolyzer, and a concentrator device configured to separate the aqueous fraction from the solid fraction of said aqueous effluents processed by means of the supercritical water reactor is electrically powered by electrical energy generated by a photovoltaic field.

10. The method according to claim 1, wherein said supercritical water reactor comprises one of:
a supercritical water oxidation reactor,
a supercritical water gasification reactor.
